(19) United States
(12) Patent Application Publication
(10) Pub. No.: US 2007/0265636 A1
(43) Pub. Date: Nov. 15, 2007

(54) Lens delivery system

(75) Inventor: Huong K. Huynh, Arlington, TX (US)

Correspondence Address: ALCON, IP LEGAL, TB4-8, 6201 SOUTH FREEWAY, FORT WORTH, TX, 76134, US

(73) Assignee: Alcon, Inc.

(21) Appl. No.: 11/431,727

(22) Filed: May 10, 2006

(51) Int. Cl. A61F 9/00; A61F 2/16

(52) U.S. Cl. 606/107; 623/6.12

(57) ABSTRACT

A lens delivery system having a cartridge and a handpiece with a lockout feature. The lockout feature helps to ensure that the appropriate combination of cartridge and handpiece is used.

This invention relates to intraocular lenses (IOLs) and more particularly to devices use to inject IOLs into an eye.

### BACKGROUND OF THE INVENTION

The human eye in its simplest terms functions to provide vision by transmitting and refracting light through a clear outer portion called the cornea, and further focusing the image by way of the lens onto the retina at the back of the eye. The quality of the focused image depends on many factors including the size, shape and length of the eye, and the shape and transparency of the cornea and lens.

When trauma, age or disease cause the lens to become less transparent, vision deteriorates because of the diminished light which can be transmitted to the retina. This deficiency in the lens of the eye is medically known as a cataract. The treatment for this condition is surgical removal of the lens and implantation of an artificial lens or IOL.

While early IOLs were made from hard plastic, such as polymethylmethacrylate (PMMA), soft, foldable IOLs made from silicone, soft acrylics and hydrogels have become increasingly popular because of the ability to fold or roll these soft lenses and insert them through a smaller incision. Several methods of rolling or folding the lenses are used. One popular method is an injector cartridge that folds the lenses and provides a relatively small diameter lumen through which the lens may be pushed into the eye, usually by a soft tip plunger. The most commonly used injector cartridge design is illustrated in U.S. Pat. No. 4,681,102 (Bartell), and includes a split, longitudinally hinged cartridge. Similar designs are illustrated in U.S. Pat. Nos. 5,494,484 and 5,499,987 (Feingold) and 5,616,148 and 5,620,450 (Eagles, et al.). In an attempt to avoid the claims of U.S. Pat. No. 4,681,102, several solid cartridges have been investigated, see for example U.S. Pat. No. 5,275,604 (Rheinish, et al.) and U.S. Pat. No. 5,653,715 (Reich, et al.).

These devices all anticipate a lens of relatively uniform size. As a result, the bore through the cartridge and the plunger used to express the lens out of the cartridge all have specific dimensions, allowing any cartridges and handpieces from the same manufacturer to be used interchangeably. Cartridges and handpieces from the same manufacturer, but of different sizes, may not be interchangeable. For example, a handpiece having a relatively large plunger tip may not be suitable for use with a cartridge having a relatively small bore.

Accordingly, a need continues to exist for a means to assure that non-interchangeable cartridges and handpieces are not used inadvertently.

### BRIEF SUMMARY OF THE INVENTION

The present invention improves upon prior art by providing a lens delivery system having a cartridge and a handpiece with a lockout feature. The lockout feature helps to ensure that the appropriate combination of cartridge and handpiece is used.

It is accordingly an objective of the present invention to provide a lens delivery system that is suitable for folding lenses made from a soft acrylic material.

It is a further objective of the present invention to provide a lens delivery system having a lockout feature.

Other objectives, features and advantages of the present invention will become apparent with reference to the drawings, and the following description of the drawings and claims.

### BRIEF DESCRIPTION OF THE DRAWINGS

FIG. 1 is an enlarged side elevational view of a cartridge that may be used with the lens delivery system of the present invention.

FIG. 2 is an enlarged partial side elevational view of a handpiece that may be used with the lens delivery system of the present invention.

FIG. 3 is an enlarged side elevational view of the cartridge illustrated in FIG. 1 installed in the handpiece illustrated in FIG. 2.

### DETAILED DESCRIPTION OF THE INVENTION

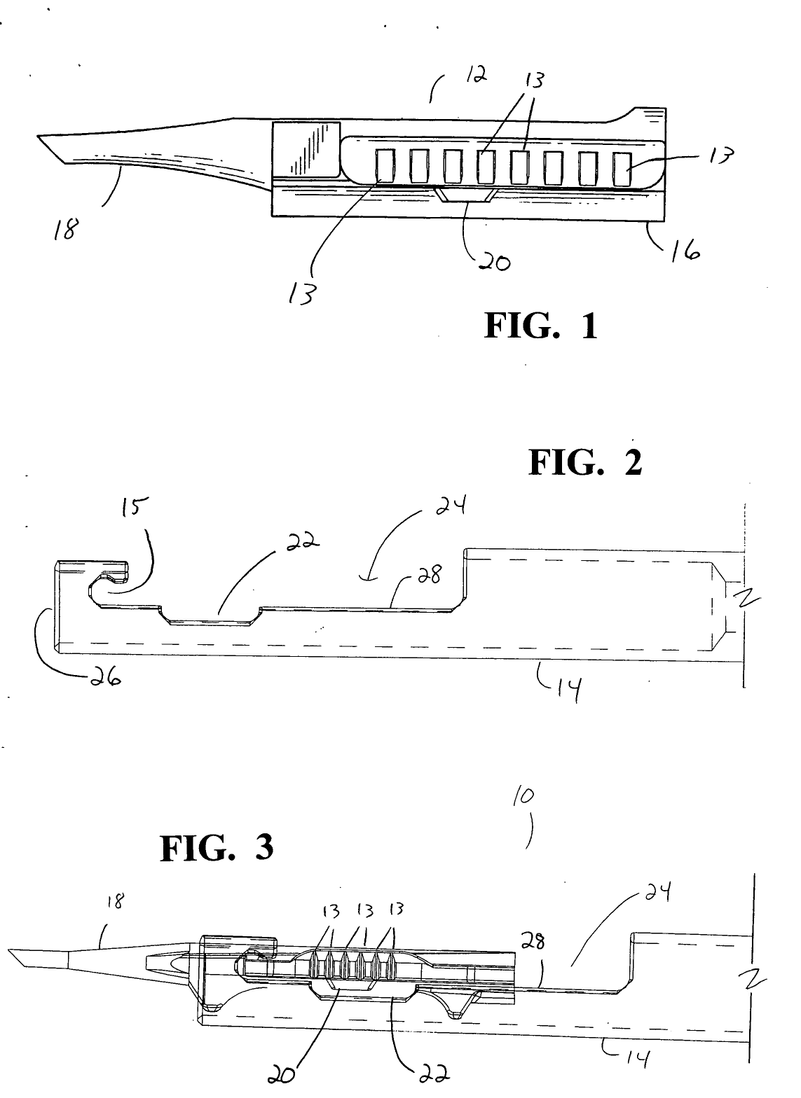

As best seen in FIG. 1, lens delivery system 10 of the present invention generally includes cartridge 12 and handpiece 14. As best seen in FIG. 1 cartridge 12 generally has tubular body 16 and injection nozzle 18. Cartridge 12 is molded as a single piece from any suitable thermoplastic, such as polypropylene, and the thermoplastic may contain a lubricity enhancing agent such as those disclosed in U.S. Pat. No. 5,716,364, the entire contents of which are incorporated herein by reference. Alternatively, cartridge 12 may be made from stainless steel or titanium. Nozzle 18 preferably is round, oval or elliptical in cross-section and has a cross-sectional area of between around 2.2 mm² to around 2.6 mm². Body 16 preferably contains grips 13 that allow easier manipulation of cartridge 12 and provide a mechanism to lock cartridge 12 within notches 15 in handpiece 14. Body 16 also contains lockout tabs 20 that interact with lockout slots 22 in handpiece 14 in the manner described below.

Handpiece 14 may be of any suitable construction, such as molded thermoplastic or machined aluminum, stainless steel or titanium. Handpiece 14 is generally tubular and contains recess 24 near distal end 26 sized and shaped to receive cartridge 12, as shown in FIG. 3. Recess 24 has generally flat sides 28 to align the longitudinal access of cartridge 12 with the longitudinal axis of handpiece 14 in a generally coaxial fashion, however; lockout tabs 22 prevent cartridge 12 from fitting within recess 24 in the absence of lockout slots 22. Lockout slots 22 are slightly longer than lockout tabs 20 to allow grips 13 on cartridge 12 to be slid within notches 15 on handpiece 14. One skilled in the art will recognize that lockout tabs 20 and lockout slots 22 can vary in shape and size so as to allow or prevent any cartridge 12/handpiece 14 combination.

While certain embodiments of the present invention have been described above, these descriptions are given for purposes of illustration and explanation. Variations, changes, modifications and departures from the systems and methods disclosed above may be adopted without departure from the scope or spirit of the present invention.

We claim:

1. An intraocular lens delivery system, comprising:
a) a cartridge;
b) a handpiece having a recess into which the cartridge can be received; and
c) a lockout feature associated with the cartridge and the handpiece to either allow or disallow the cartridge from being received in the recess of the handpiece.

2. The lens delivery system of claim 1 wherein the lockout feature comprises lockout tabs formed on the cartridge and associated lockout slots in the recess.
